(19) United States
(12) Patent Application Publication
(10) Pub. No.: US 2018/0311194 A1
(43) Pub. Date: Nov. 1, 2018

(54) APPLICATION OF TRANILAST IN PREPARATION OF DRUG TREATING SCLEREDEMA DIABETICORUM

(71) Applicant: THE FIRST AFFILIATED HOSPITAL OF NANJING MEDICAL UNIVERSITY, Nanjing (CN)

(72) Inventors: Maihua HOU, Nanjing (CN); Xinzheng LU, Nanjing (CN); Min SUN, Nanjing (CN); Feng YANG, Nanjing (CN)

(21) Appl. No.: 16/029,136

(22) Filed: Jul. 6, 2018

Related U.S. Application Data
(63) Continuation-in-part of application No. PCT/CN2017/075507, filed on Mar. 3, 2017.

(30) Foreign Application Priority Data
Jan. 6, 2016 (CN) 201610004322.2

(51) Int. Cl. A61K 31/192; A61P 17/00

(52) U.S. Cl. CPC A61K 31/192; A61P 17/00

(57) ABSTRACT

Provided is an application of tranilast in the preparation of a medicament for treating scleredema diabeticorum. Tranilast was originally used clinically for the treatment of allergic diseases and keloids, which is a safe and effective medicament with few adverse reactions that has been used clinically for many years, it is currently used in patients with scleredema diabeticorum who have no definite therapeutic schedule, and after 3 months of application, the symptoms may be observed to be significantly relieved in clinical practice, the thickness of the skin was significantly thinner detected by B-ultrasound and no significant adverse reactions were observed. This suggests that the medicament can quickly control the skin lesions of the patients with scleredema diabeticorum, reduce the clinical symptoms, and has good tolerance, high patient compliance, rare adverse reactions, and good clinical efficacy and safety.

### CROSS-REFERENCE TO RELATED APPLICATIONS

The instant application is a continuation-in-part of international application no. PCT/CN2017/075507 filed on Mar. 3, 2017, and claims priority to Chinese patent application No. 201610004322.2 filed on Jan. 6, 2016, and the entire contents of these applications are hereby incorporated by reference.

### TECHNICAL FIELD

The present invention belongs to the field of medicine, and particularly relates to an application of tranilast in the preparation of a medicament for treating scleredema diabeticorum.

### BACKGROUND ART

Scleredema diabeticorum is a rare disease characterized by diffuse symmetric skin hardening associated with diabetes. In most cases, progressive symmetric diffuse skin hardening often occurs on the back of the neck and shoulders, and it may be extended to the face, front of the neck, scalp, thoracic dorsal and upper arm in some cases. In some cases, the abdominal wall, buttocks and chest, etc. may also be affected. The skin is substantially hardened with nonpitting, normal dermatoglyph disappeares, and the surface is red and has an unclear border with normal skin. The local skin feels as usual, with the level of scleredema often the most severe in the neck, shoulders and back, and has a hard elephant skin feel when touched. If the neck is involved, it is inconvenient to turn around; and if the chest wall is involved, then inspiration chest expansion is limited. A few cases may have hepatomegaly, and skeletal muscle and myocardial involvement. Patients with diabetes often have persistent lesions. The histopathological manifestations of this disease are that: epidermis is normal, dermis is approximately 3 to 4 times thicker than normal, collagen bundles are thickened and are separated by a clear lacuna in which non-sulfate acid mucopolysaccharide deposits is confirmed, peripheral blood vessels are mildly infiltrated, most cutaneous appendages do not atrophy, and through electron microscopy, it can be observed that proliferated collagen fibers are uniform in size and arranged in a bundle with accumulation of excessive microfibrils and fibroblasts with small volume and inactive function. This condition lacks specific effective therapy. Common symptomatic treatment, control of blood glucose cannot alleviate the condition.

Tranilast was first developed in the 1970s by the Japanese, such as Eda, which is an anaphylactic mediator retardant that inhibits the mast cell degranulation and release reaction of anaphylactic mediator caused by allergen and other stimulus, has the effect of stabilizing the cell membrane of and basophilic granulocyte, to prevent them from degranulation, thus inhibiting the release of histamine and 5-hydroxytryptamine anaphylactic reaction substances, and has a significant inhibitory effect on IgE antibody-induced cutaneous anaphylaxis and experimental asthma in rats.

### SUMMARY OF THE INVENTION

An object of the present invention is to provide a new use of tranilast, ie, a new application in pharmaceuticals.

The present invention relates to an application of tranilast in the preparation of a medicament for treating scleredema diabeticorum.

There is currently no special effective therapeutic schedule for adult scleredema, scleredema diabeticorum often persists, and cannot be relieved by controlling blood glucose. Some patients may be affected by trunk movement ability and breathing due to continued progression of the disease. The use of glucocorticoids and immunosuppressors for severe patients in the world has an unreliable efficacy, and may further cause serious adverse reactions, and aggravate diabetes. Tranilast was originally used clinically for the treatment of allergic diseases and keloids, which is a safe and effective medicament with few adverse reactions that has been used clinically for many years, it is currently used in patients with scleredema diabeticorum who have no definite therapeutic schedule, after 3 months of application, the symptoms may be observed to be significantly relieved in clinical practice, the thickness of the skin was significantly thinner detected by B-ultrasound and no significant adverse reactions were observed. This suggests that the medicament can quickly control the skin lesions of the patients with scleredema diabeticorum, reduce the clinical symptoms, and has good tolerance, high patient compliance, rare adverse reactions, and good clinical efficacy and safety.

One aspect of the current invention relates to a method treating a subject having scleredema diabeticorum. The method includes administering to the subject a composition comprising an effective amount of tranilast or a pharmaceutically acceptable salt thereof. The tranilast or a pharmaceutically acceptable salt thereof is the sole active agent administered to the subject to treat the subject for scleredema diabeticorum.

The subject has been diagnosed with scleredema diabeticorum. It also can be diagnosed with diabetes. The subject can be human.

The amount of the tranilast or a pharmaceutically acceptable salt thereof administered to the subject ranges from 1 mg/day to 3 g/day, from 10 mg/day to 1 g/day, or from 0.1 g/day to 1 g/day. Alternatively, the amount of the tranilast or a pharmaceutically acceptable salt thereof administered to the subject is 0.3 g/day.

The composition can be orally administered to the subject. Preferably, the composition is administered to the subject 3 times per day.

The composition can be administered to the subject for a period of time ranging from 1 day to 6 months or from 10 day to 3 months. Alternatively, the composition is administered to the subject for a period of 2 months.

In one embodiment, the administering results in at least a reduction in the skin thickness at areas affected by scleredema diabeticorum. In another embodiment, the administering results in at least a reduction in type I collagen expression or results in at least a reduction in transforming growth factor β-induced type I collagen expression.

Another aspect of the current invention relates to a method of treating a subject having scleredema diabeticorum, the method comprising administering to the subject a composition comprising an effective amount of an inhibitor of type I collagen expression.

The details of the invention are set forth in the drawing and the description below. Other features, objects, and advantages of the invention will be apparent to those persons skilled in the art upon reading the drawing and the description, as well as from the appended claims.

### BRIEF DESCRIPTION OF THE DRAWINGS

FIG. 1 shows B-ultrasonography of a patient before medication in an example. The echo of the skin on the back of the neck was uneven, and the local echo was slightly lower, the thickness of the thickest part of the skin on the left side was about 8.7 mm, the thickness of the thickest part of the skin on the right side was about 7.5 mm, and the thickness of the surrounding skin was 3.6 mm.

FIG. 2 shows B-ultrasonography of the patient after 3 months of medication in the example. The echo of the skin on the back of the neck was uneven, and the local echo was slightly lower, the thickness of the thickest part of the skin on the left side was about 6.8 mm, the thickness of the thickest part of skin on the right side was about 6.3 mm, the thickness of the surrounding skin was 3.0 mm, and no obvious lump image was observed under the skin.

FIG. 3 is a photograph of an affected part of the patient before medication in the example.

FIG. 4 is a photograph of the affected part of the patient after 3 months of medication in the example.

### DETAILED DESCRIPTION OF THE INVENTION

The following specific clinical application of tranilast in the treatment of scleredema diabeticorum is illustrated below as an indication that tranilast may be used as a medicament for treating scleredema diabeticorum.

The used tranilast has a molecular formula:

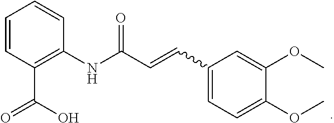 [SMILES: COc1ccc(C=CC(=O)Nc2ccccc2C(=O)O)cc1OC]

It has a specification of 100 mg per granule.

It is well established that histopathology of pathogenic skin in patients with scleredema diabeticorum shows collagen fiber thickening and type I collagen metabolism abnormalities. In our inspection, a higher level of transforming growth factor (TGF)-β can be seen by immunohistochemistry in patients with scleredema diabeticorum compared with healthy patients. It has been reported that tranilast was able to suppress the TGF-β-induced upregulation of type I collagen mRNA expression and the basal level of type I collagen mRNA in human dermal fibroblasts (Chung K Y, Kang D S, Regulation of type I collagen and interstitial collagenase mRNA expression in human dermal fibroblasts by colchicine and D-penicillamine. Yonsei Med J 1999; 40:490-495).

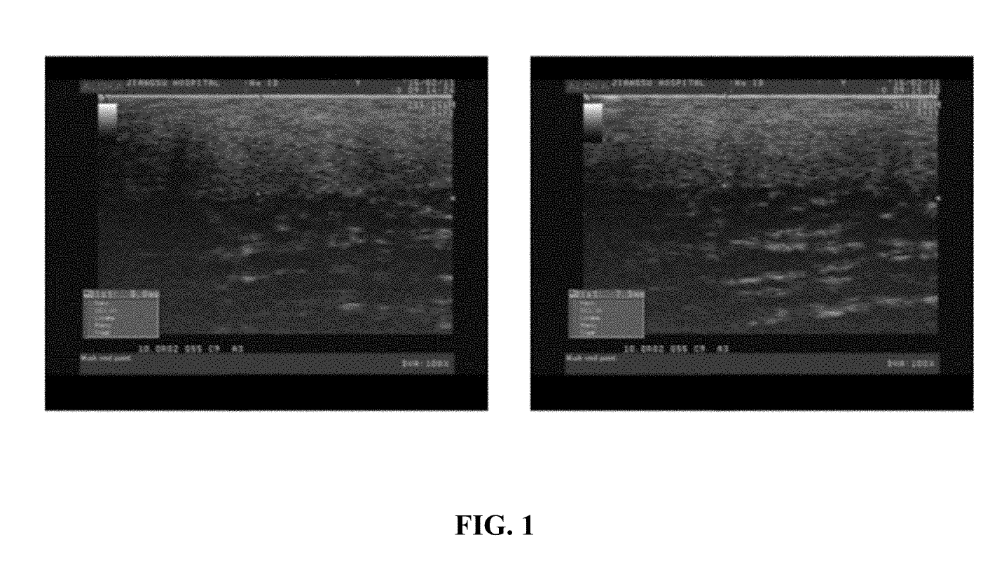

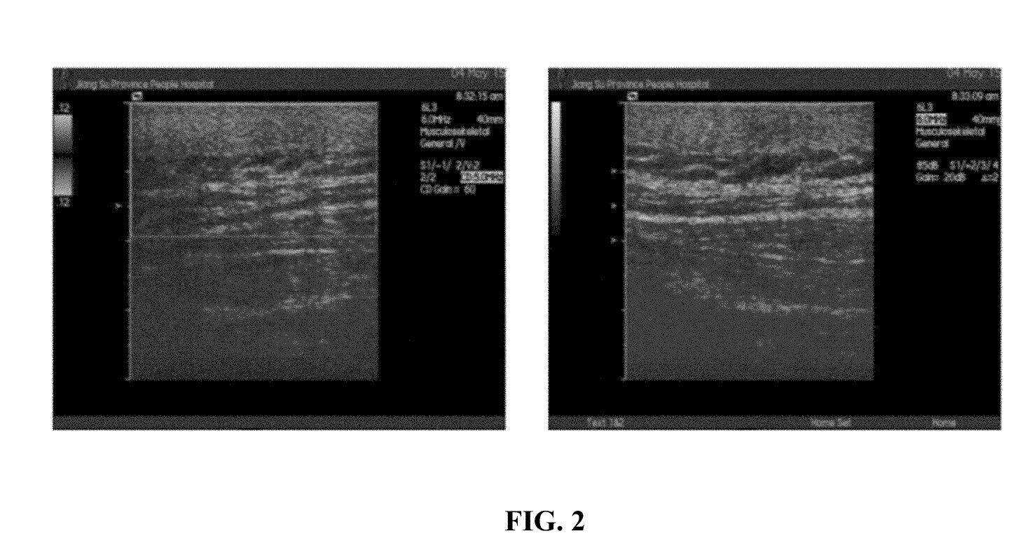

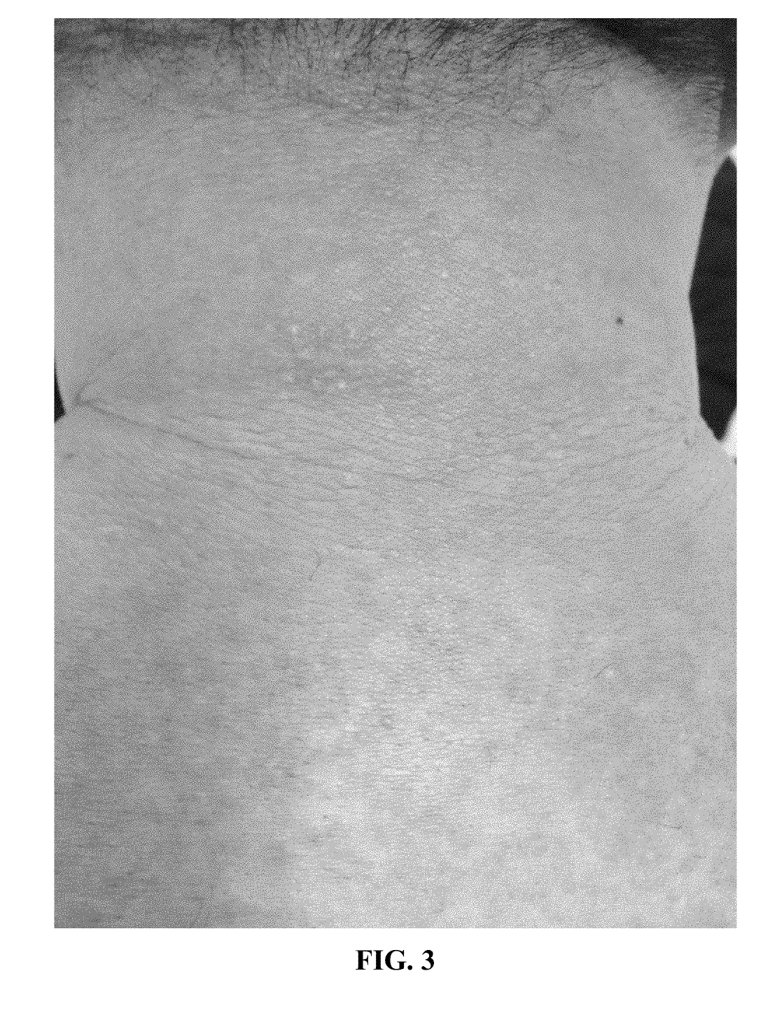

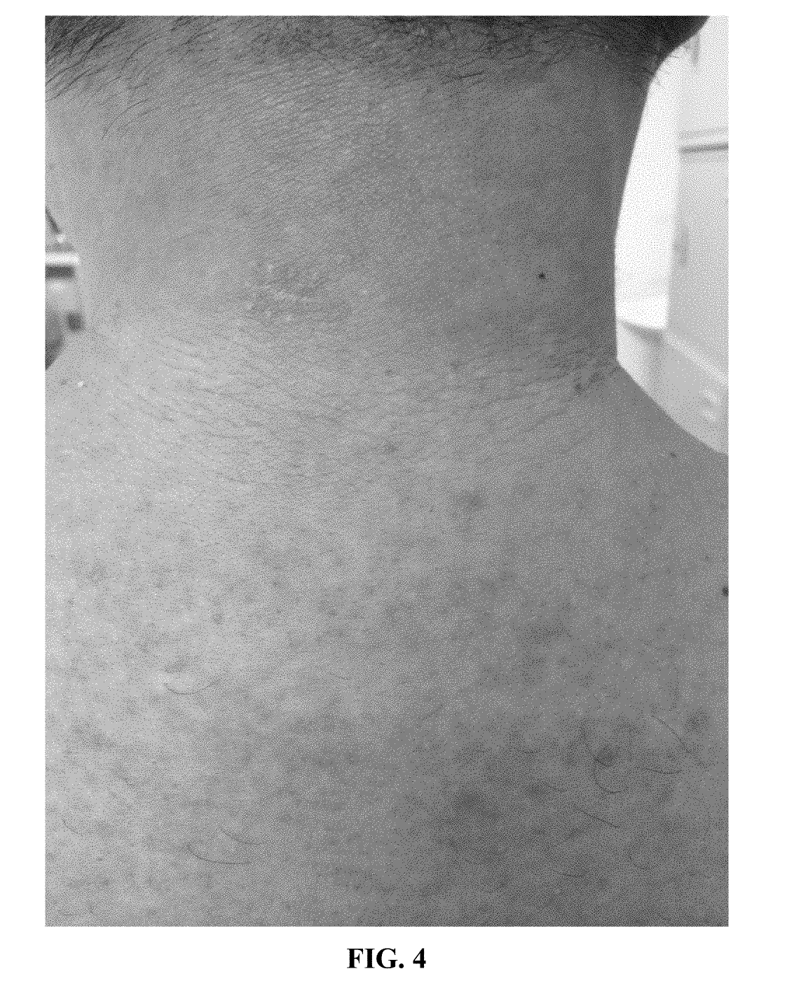

Among the 3 patients, 2 patients were males in their 40s, and 1 patient was 60-year-old female, all had type 2 diabetes for many years and a history of scleredema diabeticorum for more than 5 years. The B-ultrasound detection revealed that the skin thickness increased in different degrees. The patients were given tranilast capsule 0.1, 3 times a day orally, and after 3 months, the local skin erythema subsided significantly, became thinner and sofer, the wrinkles formed due to thickening of the skin were significantly reduced, and the uncomfortable symptom of conscious hardening were significantly relieved. FIG. 1 is a B-ultrasonography of a male patient in his 40s before medication (a and b are different parts of the affected skin). According to the B-ultrasound examination, it was found that before the treatment, the maximum skin thickness of the patient was 7.8 to 8.7 mm, and the maximum skin thickness was reduced by 2 mm after 3 months of treatment (FIG. 2, a and b are different parts of the affected skin). There were no obvious abnormalities in blood routine examination, routine urine examination, and biochemical detection. Photographs of the affected part before and after medication are shown in FIG. 3 and FIG. 4.

The clinical results showed that tranilast has a good therapeutic effect on scleredema diabeticorum.

Supplementary Content:

1. Mode of administration: oral, tranilast 0.1 g, 3 times a day, every 3 months for a course of treatment, a total of two courses.

2. Mechanism of action: It was initially found that tranilast reduces skin thickness by inhibiting secretion and synthesis of mucin by fibroblast, and it is expected that the medicament can be used in all diseases where mucin secretion is increased.

3. Several cases were further treated, the patients all showed skin thinning, and several patients were on medication.

Example 1, female, 63 years old, with thickening of the back skin for five years, had a skin thickness of 6.5 mm measured by B-ultrasound, and after 3 months of oral administration of tranilast, had a skin thickness of 6 mm measured by B-ultrasound.

Example 2, male, 47 years old, with thickening of the back skin for 2 years, had a skin thickness of 8 mm measured by B-ultrasound, took Tranilast 0.1, three times a day, and after 3 months, had a skin thickness of 6 mm measured by B-ultrasound.

Example 3, male, 53 years old, with thickening of the back skin for 5 years, had a skin thickness of 8.5 mm measured by B-ultrasound, took Tranilast 0.1, three times a day, and after 3 months, had a skin thickness of 5.9 mm measured by B-ultrasound.

1. A method of treating a subject having scleredema diabeticorum, the method comprising administering to the subject a composition comprising an effective amount of tranilast or a pharmaceutically acceptable salt thereof.

2. The method according to claim 1, wherein the tranilast or a pharmaceutically acceptable salt thereof is the sole active agent administered to the subject to treat the subject for scleredema diabeticorum.

3. The method according to claim 1, wherein the subject has been diagnosed with scleredema diabeticorum.

4. The method according to claim 1, wherein the subject has been diagnosed with diabetes.

5. The method according to claim 1, wherein the tranilast has the formula:

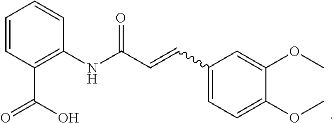 [SMILES: COc1ccc(C=CC(=O)Nc2ccccc2C(=O)O)cc1OC]

6. The method according to claim 1, wherein the subject is human.

7. The method according to claim 1, wherein the amount of the tranilast or a pharmaceutically acceptable salt thereof administered to the subject ranges from 1 mg/day to 3 g/day.

8. The method according to claim 1, wherein the amount of the tranilast or a pharmaceutically acceptable salt thereof administered to the subject ranges from 10 mg/day to 1 g/day.

9. The method according to claim 1, wherein the amount of the tranilast or a pharmaceutically acceptable salt thereof administered to the subject ranges from 0.1 g/day to 1 g/day.

10. The method according to claim 1, wherein the amount of the tranilast or a pharmaceutically acceptable salt thereof administered to the subject is 0.3 g/day.

11. The method according to claim 1, wherein the composition is orally administered to the subject.

12. The method according to claim 1, wherein the composition is administered to the subject 3 times per day.

13. The method according to claim 14, wherein the composition is administered to the subject for a period of time ranging from 1 day to 6 months.

14. The method according to claim 14, wherein the composition is administered to the subject for a period of time ranging from 10 day to 3 months.

15. The method according to claim 14, wherein the composition is administered to the subject for a period of 2 months.

16. The method according to claim 1, wherein the administering results in at least a reduction in the skin thickness at areas affected by scleredema diabeticorum.

17. The method according to claim 1, wherein the administering results in at least a reduction in type I collagen expression.

18. The method according to claim 1, wherein the administering results in at least a reduction in transforming growth factor β-induced type I collagen expression.

19. A method of treating a subject having scleredema diabeticorum, the method comprising administering to the subject a composition comprising an effective amount of an inhibitor of type I collagen expression.

20. The method according to claim 19, wherein the subject has been diagnosed with diabetes.
